(19) United States
(12) Patent Application Publication
(10) Pub. No.: US 2026/0007564 A1
(43) Pub. Date: Jan. 8, 2026

(54) METHOD FOR ADAPTING MASSAGE SEQUENCES TO DIFFERENT TYPES OF SEATS

(71) Applicant: Conti Temic microelectronic GmbH, Ingolstadt (DE)

(72) Inventors: Michael Beuschel, Stammham (DE); Stefan Bauer, Engelbrechtsmünster (DE)

(73) Assignee: Conti Temic microelectronic GmbH, Ingolstadt (DE)

(21) Appl. No.: 19/257,973

(22) Filed: Jul. 2, 2025

(30) Foreign Application Priority Data
Jul. 3, 2024 (DE) 10 2024 206 236.7

(51) Int. Cl. A61H 9/00; A47C 7/14; B60N 2/90; G16H 40/67

(52) U.S. Cl. CPC A61H 9/0007; A47C 7/142; A61H 9/0078; B60N 2/914; B60N 2/976; G16H 40/67; A61H 2201/0149; A61H 2201/1238; A61H 2201/5002; A61H 2201/5007; A61H 2201/5056; A61H 2203/0431

(57) ABSTRACT

A method for adapting massage sequences to different types of seats, which have a number of inflatable cushions arranged at specified locations in the seat, is described. Here, a calculation model is parameterized for a specific seat type, and the duration of control commands for filling and ventilation of a cushion is adapted for each step such that a specified degree of filling is achieved, wherein the total temporal length of a step may vary in order either to achieve continuous operation of the compressor or in order to adhere to pauses of a defined minimum duration in the activation of the compressor.

### CROSS-REFERENCE TO RELATED APPLICATIONS

This application claims priority to German patent application No. 10 2024 206 236.7, filed Jul. 3, 2024, which is hereby incorporated by reference.

### TECHNICAL FIELD

The technical field relates generally to filling elastic cushions of seats with a fluid to achieve a massage function.

### BACKGROUND

In transportation, fillable elastic cushions are increasingly used for the purposes of shaping seat contours. These allow the seats to be individually adapted to the occupant. For this purpose, the elastic cushions are generally filled with air. However, any other fluid or gas is also possible. Originally found in high-priced means of transportation, this equipment is now entering the middle and lower price segments.

Where such air cushions, which can be filled with compressors via controllable valves, are provided, alternating filling and ventilation processes can also be controlled in order to implement massage functions. Pneumatic massage functions normally use a plurality of pneumatic air cushions in the seat back, which are cyclically filled and emptied in order to impart a dynamic force effect similar to a manual massage. It is sought here to achieve predefined pressure sequences in the individual air cushions. At present, use is generally made of purely time-based control, that is to say without feedback from a pressure sensor. If a new massage sequence is created, an existing massage sequence is to be ported to a different seat, or the components used are changed (for example a compressor of different power or valves having a different cross section), the control times must be newly determined experimentally in each case.

DE 10 2010 063 136 B4 describes a pneumatic system in which the vessel pressure is determined by means of a mathematical model.

DE 10 2011 122 392 A1 describes a pressure model based on characteristic curves. The effect of all components of a system for filling, holding and ventilating is represented in a single characteristic curve in each case. An assignment to individual components is therefore not possible, nor is partial parameterization when changes are made to a part of the system.

DE 10 2018 209 386 B3 also describes a pressure model, which uses a simplified calculation of the physical behavior of the pneumatic components.

DE 20 2020 105 243 U1 describes a massage system in which the sequence control is executed on a central vehicle computer and can also receive updates from an external server.

A disadvantage of the last-mentioned massage system is that the temporal sequence of the massage control commands must in each case be adapted exactly to the seat type installed in the vehicle. Otherwise, different installed pneumatic components (compressor, valves, hose lines, air cushions, etc.) and also seat properties (for example upholstery, cover, seams) would lead to a different massaging effect and, under certain circumstances, also to overloading of components.

It is also expected that, in future, massage sequences will for example be able to be booked as a service, or created by end customers themselves and exchanged with other end customers. All of this increases the number of massage sequences available and consequently the (generally experimental) effort required to adapt each individual massage sequence to each possible seat type.

As such, it is desirable to provide a method for simplified adaptation of massage sequences to different seat types.

### SUMMARY

The disclosure provides a method for adapting massage sequences to different types of seats. The seats have a number of inflatable cushions arranged at specified locations. Each cushion is connected via a controllable valve assigned thereto to a controllable compressor for the purposes of filling with a fluid and to the environment for the purposes of emptying. Each valve is connected to a control unit, which is designed to control the valve utilizing control signals such that an assigned cushion is fluidically connected via the valve to the compressor for the purposes of filling or to the environment for the purposes of emptying. A parameterizable calculation model can determine the properties of the inflatable cushions, at least with regard to the time within which a specified degree of filling is achieved, depending on at least their location in the vehicle seat, the power of the compressor, and their size, in order to determine therefrom the degree of filling of a cushion. The calculation model is connected to the control unit in order to provide a signal representing the degree of filling of a cushion to the control unit. The control unit provides control signals calculated therefrom for the valves and the controllable compressor as input variables to the calculation model. The control unit determines the control signals for a valve depending on the signal for the degree of filling, determined by the calculation model, of the assigned cushion, and depending on a control signal for the compressor. The control unit is connected to the controllable compressor, and wherein at least the following steps are executed:

the calculation model is parameterized for a specific seat type, and the duration of the control commands for filling and ventilation of a cushion is adapted for each step such that a specified degree of filling is achieved, wherein the total temporal length of a step may vary in order either to achieve continuous operation of the compressor or in order to adhere to pauses of a defined minimum duration in the activation of the compressor.

A massage sequence consists of steps, which in turn each contain at least a duration and an intensity (e.g., pressure to be achieved in a cushion). Holding phases and pauses can also be specified. Optionally, a region or position specification of a cushion in a seat may also be included for each step.

All seats of a particular type use the same parts, such as cushions, compressors, valves, fluid lines, etc., for all components involved in the massage.

The calculation model is parameterized for the specific seat type. This model estimates the degree of filling of the individual massage air chambers or cushions depending on control and optionally environmental and usage conditions, which are provided to said model by the control unit and/or by sensors. The calculation model is configured to provide sufficiently accurate values even under a wide variety of seat loads (e.g., body sizes).

The target values of each step of the massage sequence are read into the control unit, and the control commands are output to the valves for the purposes of filling and emptying a cushion depending on the degree of filling estimated by the calculation model, such that the target values are achieved as closely as possible.

The calculation is performed in advance, when no massage sequence is being executed. According to the disclosure, the duration of the control commands for filling and ventilation of a cushion is adapted for each step such that a specified degree of filling is achieved, wherein the total temporal length of a step may vary.

In one embodiment of the method, in the event that fewer cushions are present in a seat type than are provided in a massage sequence, massage steps can be applied to the next nearest cushions.

In one embodiment of the method, the power of the compressor is adapted by the control unit such that the specified duration either of each individual step or of a plurality of steps is adhered to as exactly as possible.

Here, the control unit calculates the required activation durations and starts and optionally the required power, or power to be adapted, of the compressor in advance, depending on the degree of filling determined by the calculation model. This may be performed successively in cycles until an optimum characteristic has been determined, which is then implemented during operation utilizing the determined control signals.

In a further embodiment of the method, the target values of each step of a massage sequence are read into the control unit, wherein the target values of a step comprise at least the duration of a control command and the desired degree of filling of a cushion; if the compressor power is to be changed, the control unit uses the parameters of the seat type, and the input target values for the degree of filling of a cushion and the duration of a control command, to determine the compressor power required to adhere to the specified duration; if the compressor power is not to be changed during a massage sequence, the control unit uses the parameters of the seat type, the input target values for the degree of filling of a cushion, and a fixed compressor power, to determine the duration of a control command required to achieve the specified degree of filling, wherein, if the determined duration is longer than the specified duration, the start of filling should commence earlier if the earlier start falls within a pause; and the determined values for the start and the duration of a filling process and the compressor power required for a step are stored in the control unit and are used during operation for the control signals to the compressor and the valves.

In one further development of the method, the duration of a control command for filling a cushion is shortened if necessary in relation to the target value, for each step of a massage sequence, such that a specified degree of filling is not exceeded.

This can be understood as a fall-back level and can optionally also be adapted during operation.

In a further embodiment of the method, if uniform compressor running is to be implemented, the compressor power is increased or decreased for the entire massage sequence, such that the total duration of the sequence corresponds again to the specified value.

Here, for uniform compressor running, the compressor rotational speed may advantageously be kept constant.

### BRIEF DESCRIPTION OF THE FIGURES

The disclosure will be described in more detail below on the basis of exemplary embodiments and with reference to figures, in which:

FIG. 1 is a schematic illustration of a (vehicle) seat having components required for a massage function,

FIG. 2 shows a simple massage sequence according to specified target durations and target degrees of filling,

FIG. 3 shows a simple massage sequence with filling and emptying durations adapted to a different seat type by means of a calculation model and

FIG. 4 shows an application for transmitting massage sequences to different types of seats.

### DETAILED DESCRIPTION

Exemplary implementations will be described below. Where reference is made here to the degree of filling of an air cushion, the pressure or alternatively the volume, the mass of the air in the air cushion or the geometric stroke of the air cushion is also meant.

Where the term “adjustment rate” is used here, this means, for example, a pressure gradient, a compressor rotational speed, voltage or power, a volume or mass flow, and also an adjustable admission pressure.

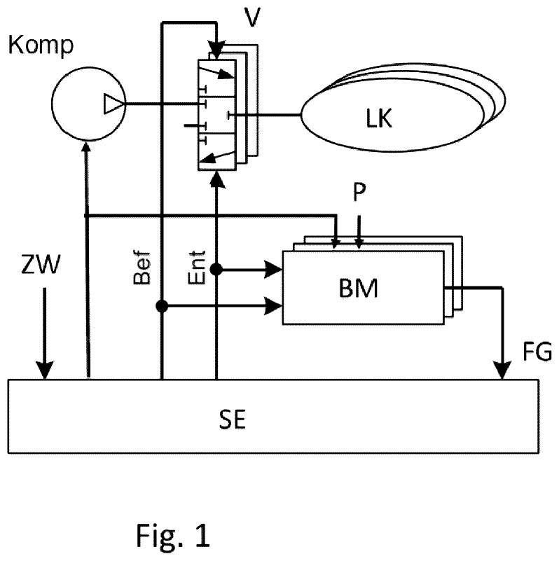

FIG. 1 shows a schematic diagram of a vehicle seat having a compressor Komp, which can pump air via valves V into air cushions LK assigned to the valves V. Where air is mentioned here, it is equally also possible for gas or some other fluid to be used or meant. The valves V are controlled by a control unit SE using control commands Bef, Ent, by means of which one of optionally a plurality of valves V can receive a filling command Bef, for the purposes of connecting an air cushion LK, which is assigned to said valve, via the valve V to the compressor Komp, or an emptying command Ent, for the purposes of connecting an air cushion LK via the assigned valve V to the environment. The control commands constitute commands which can actually be executed but which, in the method proposed here, can be used for precalculation of the ideal control commands and can be calculated successively.

The vehicle seat has a calculation model BM for the degree of filling FG of the normally several air cushions LK, which calculation model can estimate the degree of filling FG at least from the valve control times, seat parameters P and the power of the compressor KOMP, the valve control times being determined from the control commands Bef, Ent. A parameter set P is determined in advance on a one-off basis for each seat type, which parameter set can then be used in all seats of the same seat type and is input into the calculation model BM. The calculation model BM may also be part of the control unit SE and, in particular, be implemented by a program for execution in a processor. A neural network or artificial intelligence may also be used.

The target values ZW for the pressure in the cushion and/or the times for filling and emptying may be specified to the control unit SE, such that the control commands Bef, Ent can also be executed accordingly depending on the degree of filling FG determined by the calculation model BM.

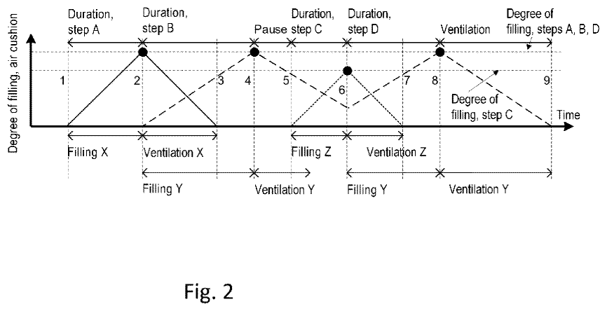

FIG. 2 shows an example of a short massage sequence with 4 steps, wherein a (target) duration and a (target) degree of filling is defined for each step. Pauses may additionally also be incorporated. Different values may be specified for each step and each air cushion LK, depending on the typical or expected behavior of the seat, the air cushion LK, the valves V and the air supply (compressor KOMP) and depending on the desired massage effect.

The individual steps are as follows:
Step A: At time (1), air cushion X should start filling.
Step B: As soon as air cushion X has achieved the target degree of filling (time 2), ventilation of said air cushion (until time 3) starts on the basis of control by the control unit SE. At the same time, air cushion Y starts filling, in order that the compressor KOMP can run without interruption.
Pause: As soon as air cushion Y has achieved the target degree of filling (time 4), ventilation of said air cushion starts on the basis of control by the control unit SE. Until the next step, a pause is introduced, during which, for example, the compressor KOMP is at rest (times 4 to 5).
Step C: Air cushion Z is filled to a lower specified target degree of filling (times 5 to 6).
Step D: Renewed filling of air cushion Y starts immediately thereafter (time 6). At this time, said air cushion has not yet been fully ventilated. The time period (6 to 8) is therefore specified to be shorter than in step B (times 2 to 4).

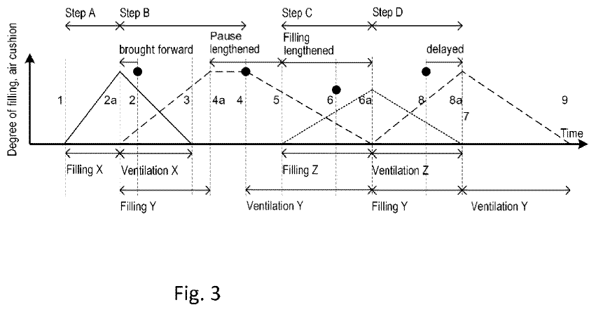

FIG. 3 now illustrates an example describing, for another seat type and thus for other values (parameters) for the valve and air line cross sections, compressor power, etc., how the degrees of filling estimated by means of a calculation model BM affect the control commands derived therefrom by the control unit SE:
Step A: Air cushion X is filled faster than specified. This brings the end of the filling process forward from time (2) to time (2a).
Step B: In order to avoid an excessively short pause (from time 2a to time 2) for the compressor KOMP, the start of step B is also brought forward to time (2a). Air cushion Y also fills faster than specified (until time 4a).
Pause: The pause is extended (from time 4a to time 5) in order to be synchronized with the specified time again at time (5).
Step C: Air cushion Z fills more slowly than specified. This delays the end of the filling process from time (6) to time (6a).
Step D: Accordingly, air cushion Y can be filled again only starting from time (6a). The calculation model BM takes into account that air cushion Y has already been fully ventilated at this point. The time at which the target degree of filling is achieved is thus shifted (despite faster filling) from time (8) to time (8a).

The filling and ventilation sequences of the air cushions X, Y, Z as shown in FIG. 3, which are based on control commands for valves V assigned to the air cushions X, Y, Z, show a possible characteristic of the method in online operation. In this case, it is possible to react to deviations of the seat type from the specified target values retrospectively-after the calculation of the degree of filling FG by the calculation model BM—by virtue of times being shifted, for example. The method is however also suitable for offline calculation, in which a change in the compressor power can also be taken into consideration. It is possible here for the ideal compressor power to be determined and set or stored in advance for each step, in order to achieve the specified degree of filling and the specified duration as exactly as possible.

It would thus be possible to reduce the compressor power for air cushion X in order to adhere to the desired duration for step A. It would analogously also be possible for the compressor power to be increased in step C.

Since the slower filling of air cushion Z is identified in the offline calculation, the control command for filling at time (5) can instead also be brought forward toward time (4) in order to achieve the target degree of filling again, as specified, at time (6) without the need to change the compressor power.

If uniform compressor running (for example a constant rotational speed) is desired, it is also possible for the compressor power to be increased or decreased for the entire sequence, such that the total duration of the sequence corresponds again to the specified value; here, individual steps may however then be longer or shorter than specified.

Using the method described above, massage programs can be freely exchanged between seats that have the same number of air cushions and a similar arrangement thereof.

By means of an optional region or position specification (for example by means of uniform coordinates within the seat) for each step, massage programs can additionally also be executed on seats having a different number or arrangement of air cushions. Here, all steps are assigned to the air cushions that are situated closest in terms of their position. It is also possible to skip steps for positions which are not present.

For example, a wave-like massage sequence (that is to say air cushions being filled in succession in a vertical direction) can thus be adapted to the actual number of air cushions present. If 6 air cushions (numbers 1 to 6) are specified in such a massage sequence, but the seat has only 4 air cushions, the following assignment may for example be implemented:
Air cushion specification 1→air cushion 1 in the seat
Air cushion specification 2→air cushion 2 in the seat
Air cushion specification 3-skip.
Air cushion specification 4→air cushion 3 in the seat
Air cushion specification 5-skip
Air cushion specification 6→air cushion 4 in the seat

It is possible to choose to maintain the (average) duration of each step or the total duration of the sequence.

Thus, in offline operation, combinations of optimization criteria are also possible, for example by virtue of necessary changes in adjustment rate, step lengths and pauses or deviations in relation to the target values thereof being weighted with respective factors and optimized collectively.

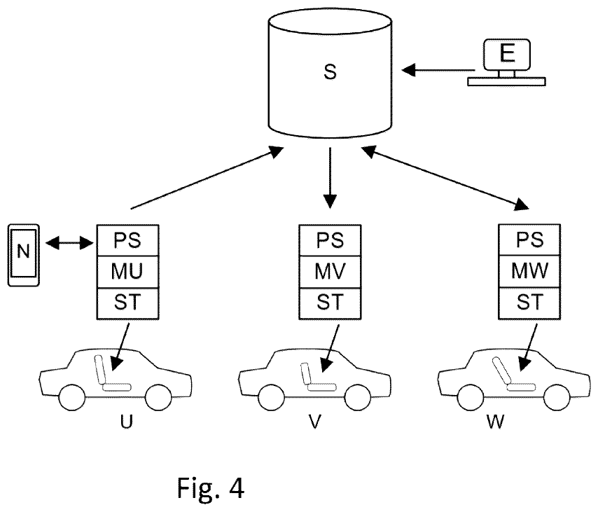

FIG. 4 shows an example of possible applications of the method for different usage situations. An external memory (or server) S in the form of a database is connected to a plurality of vehicles, in which seats of different types U, V, W are installed. Universally applicable massage programs are stored in the database.

For each seat or each vehicle, there is a program memory PS for storing one or more universally applicable massage programs. In addition, each vehicle has a calculation model MU, MV, MW, which is parameterized for the specific seat type U, V, W. Finally, a controller ST executes the resulting control commands on the specific seat.

FIG. 4 shows an exemplary application. Different usage scenarios are conceivable here, some of which will be described below:
The user N of the vehicle with seat type U creates or edits their own massage program in their program memory PS and uploads it to the server S. On their own seat, the massage program is adapted to the seat type U by the calculation model MU and is executed by the controller ST.
The user of the vehicle with seat type V downloads massage programs from the server S to their program memory PS (for example including that created by user N). On their own seat, the massage program is adapted to the seat type V by the calculation model MV and is executed by the controller ST.
The user of the vehicle with seat type W downloads massage programs from the server S and uploads them to the server S. On their own seat, the massage program is adapted to the seat type W by the calculation model MW and is executed by the controller ST.
The vehicle manufacturer E (or else a fleet operator or service provider) creates further universal massage programs and stores them on the server S in order that users of different vehicles can download them to their program memory PS.

The aforementioned embodiments have the advantage of providing improved exchangeability of massage programs between seats of different types, with new configuration options being made possible for end customers, for their vehicles, by way of individually selected massage programs.

It is no longer necessary to adjust the massage programs to the seat types used (or to the vehicle/seat manufacturer). This significantly reduces the development effort for new massage programs.

This protects the pneumatic components against overloading in the case of user-defined massage programs or those created by third parties.

1. A method for adapting massage sequences to different types of seats, which have a number of inflatable cushions arranged at specified locations in the seat,
wherein each cushion is connected via a controllable valve assigned thereto to a controllable compressor for the purposes of filling with a fluid and to the environment for the purposes of emptying,
wherein each valve is connected to a control unit, which is designed to control the valve with control signals such that an assigned cushion is fluidically connected via the valve to the compressor for the purposes of filling or to the environment for the purposes of emptying,
utilizing a parameterizable calculation model, which can determine the properties of the inflatable cushions, at least with regard to the time within which a specified degree of filling is achieved, depending on at least their location in the seat, the power of the compressor and their size, in order to determine therefrom the degree of filling of a cushion, wherein the calculation model is connected to the control unit in order to provide a signal representing the degree of filling of a cushion to the control unit, and the control unit provides control signals calculated therefrom for the valves and the controllable compressor as input variables to the calculation model,
wherein the control unit determines the control signals for a valve depending on the signal for the degree of filling, determined by the calculation model, of the assigned cushion and depending on a control signal for the compressor, wherein the control unit is connected to the controllable compressor, and wherein at least the following steps are executed:
the calculation model is parameterized for a specific seat type,
the duration of the control commands for filling and ventilation of a cushion is adapted for each step such that a specified degree of filling is achieved, wherein the total temporal length of a step may vary in order either to achieve continuous operation of the compressor or in order to adhere to pauses of a defined minimum duration in the activation of the compressor.

2. The method as claimed in claim 1, in which the power of the compressor is adapted by the control unit such that the specified duration either of each individual step or of a plurality of steps is adhered to as exactly as possible.

3. The method as claimed in claim 1, in which the target values of each step of a massage sequence are read into the control unit, wherein the target values of a step comprise at least the duration of a control command and the desired degree of filling of a cushion, if the compressor power is to be changed, the control unit uses the parameters of the seat type, and the input target values for the degree of filling of a cushion and the duration of a control command, to determine the compressor power required to adhere to the specified duration,
if the compressor power is not to be changed during a massage sequence, the control unit uses the parameters of the seat type, the input target values for the degree of filling of a cushion, and a fixed compressor power, to determine the duration of a control command required to achieve the specified degree of filling, wherein, if the determined duration is longer than the specified duration, the start of filling should commence earlier if the earlier start falls within a pause,
the determined values for the start and the duration of a filling process and the compressor power required for a step are stored in the control unit and are used during operation for the control signals to the compressor and the valves.

4. The method as claimed in claim 1, in which the duration of a control command for filling a cushion is shortened if necessary in relation to the target value, for each step of a massage sequence, such that a specified degree of filling is not exceeded.

5. The method as claimed in claim 1, in which, if uniform compressor running is to be implemented, the compressor power is increased or decreased for the entire massage sequence, such that the total duration of the sequence corresponds again to the specified value.

6. The method as claimed in claim 5, in which, for uniform compressor running, the compressor rotational speed is kept constant.

7. The method as claimed in claim 1, in which the calculation model estimates the degree of filling of the individual cushions also depending on environmental and usage conditions.
